SEQUENCE LISTING (3 sequences)

SEQ ID NO: 1 - DNA, 1428 bp, Lactobacillus rhamnosus
ctcgctccctaaaagggttacgccaccggcttcgggtgttacaaactctcatggtgtgacgggcggtgtgtacaaggcccgggaacgtattcaccgcggcgtgctgatccgcgattactagcgattccgacttcgtgtaggcgagttgcagcctacagtccgaactgagaatggctttaagagattagcttgacctcgcggtctcgcaactcgttgtaccatccattgtagcacgtgtgtagcccaggtcataaggggcatgatgatttgacgtcatccccaccttcctccggtttgtcaccggcagtcttactagagtgcccaactaaatgctggcaactagtcataagggttgcgctcgttgcgggacttaacccaacatctcacgacacgagctgacgacaaccatgcaccacctgtcattttgcccccgaaggggaaacctgatctctcaggtgatcaaaagatgtcaagacctggtaaggttcttcgcgttgcttcgaattaaaccacatgctccaccgcttgtgcgggcccccgtcaattcctttgagtttcaaccttgcggtcgtactccccaggcggaatgcttaatgcgttagctgcggcactgaagggcggaaaccctccaacacctagcattcatcgtttacggcatggactaccagggtatctaatcctgttcgctacccatgctttcgagcctcagcgtcagttacagaccagacagccgccttcgccactggtgttcttccatatatctacgcatttcaccgctacacatggagttccactgtcctcttctgcactcaagtttcccagtttccgatgcacttcctcggttaagccgagggctttcacatcagacttaaaaaaccgcctgcgctcgctttacgcccaataaatccggataacgcttgccacctacgtattaccgcggctgctggcacgtagttagccgtggctttctggttggataccgtcacgccgacaacagttactctgccgaccattcttctccaacaacagagttttacgacccgaaagccttcttcactcacgcggcgttgctccatcagacttgcgtccattgtggaagattccctactgctgcctcccgtaggagtttgggccgtgtctcagtcccaatgtggccgatcaacctctcagttcggctacgtatcattgccttggtgagccgttacctcaccaactagctaatacgccgcgggtccatccaaaagcgatagcttacgccatctttcagccaagaaccatgcggttcttggatttatgcggtattagcatctgtttccaaatgttatcccccacttaagggcaggttacccacgtgttactcacccgtccgccactcgttcaaaattaaatcaagatgcaagcacctttcaataatcagaactcgttcga

SEQ ID NO: 2 - DNA, 20 bp, Artificial
  PCR primer 27f
agagtttgatcctggctcag

SEQ ID NO: 3 - DNA, 21 bp, Artificial
  PCR primer 1492r
tacggctaccttgttacgact

(19) United States
(12) Patent Application Publication
(10) Pub. No.: US 2021/0127712 A1
(43) Pub. Date: May 6, 2021

(54) LACTOBACILLUS RHAMNOSUS STRAIN 753 AND USES THEREOF, SILAGE ADDITIVE AND SILAGE

(71) Applicant: SICHUAN AGRICULTURAL UNIVERSITY, Chengdu (CN)

(72) Inventors: Xinquan ZHANG, Chengdu (CN); Hao GUAN, Chengdu (CN); Yanhong YAN, Chengdu (CN); Yang SHUAI, Chengdu (CN); Xiaoling LI, Chengdu (CN); Qifan RAN, Chengdu (CN); Gang NIE, Chengdu (CN); Xia WANG, Chengdu (CN); Ting HUANG, Chengdu (CN); Dandan LI, Chengdu (CN); Zhongfu YANG, Chengdu (CN); Xiao MA, Chengdu (CN); Linkai HUANG, Chengdu (CN)

(73) Assignee: SICHUAN AGRICULTURAL UNIVERSITY, Chengdu (CN)

(21) Appl. No.: 17/086,381

(22) Filed: Oct. 31, 2020

(30) Foreign Application Priority Data
Nov. 1, 2019 (CN) 201911058479.3

(51) Int. Cl. A23K 30/18; C12R 1/225; A23K 10/30; A23K 10/18

(52) U.S. Cl. CPC A23K 30/18; C12R 1/225; A23Y 2220/73; A23K 10/18; A23K 10/30

(57) ABSTRACT

Disclosed are Lactobacillus rhamnosus strain 753 and uses thereof, a silage additive and silage. Lactobacillus rhamnosus strain 753 is deposited in China General Microbiological Culture Collection Center with an accession number of CGMCC 18233. Lactobacillus rhamnosus strain 753 can improve the quality of silage in a high-temperature and high-humidity region, and the silage processed by Lactobacillus rhamnosus strain 753 has good stability and low pH, low aflatoxin B1 content and less dry matter loss. In addition, secondary fermentation can be avoided in the silage processed by Lactobacillus rhamnosus strain 753 when a silo or bale for silage is opened.

### CROSS REFERENCE TO RELATED APPLICATIONS

This application claims the priority of Chinese Patent Application No. 201911058479.3, filed on Nov. 1, 2019, and the disclosures of which are hereby incorporated by reference.

### SEQUENCE LISTING

Sequence Listing is being submitted as an ASCII text file via EFS-Web, file name “200082-APXU-SICAU-Sequence-Listing.txt”, size 3 KB, created on 10/30/2020, the content of which is incorporated herein by reference.

### FIELD

The present disclosure relates to the technical field of agriculture, and in particular relates to a strain 753 of Lactobacillus rhamnosus and uses thereof, a silage additive and silage.

### BACKGROUND

Ensiling is the conversion of carbohydrates to organic acids under the anaerobic fermentation of lactic acid bacteria, thereby lowering the pH for long-term storage. Lactic acid bacteria and temperature are the determining factors for the quality of silage fermentation.

The natural ensiling is to ferment using lactic acid bacteria existing on natural plants, but the content of the lactic acid bacteria on the natural plants is low and only accounts for 0.01-1% of the total number of bacteria. Therefore, during the fermentation process, the lactic acid bacteria hardly form a dominant bacterial flora rapidly, and the pH value in the materials cannot be reduced in a short time. As a result, 1) the growth and reproduction of aerobic microorganism cause the temperature to rise rapidly, prolonging the pre-fermentation time; 2) in the process of pre-fermentation, a large amount of nutrient components and energy are loss due to the high temperature, and it also causes pungent smell and poor palatability; 3) the mass propagation of mold and putrefying bacteria in the fermentation process causes mildewing and rotting of the silage, especially on the top, the bottom and the edges of the silage; 4) because of the existence of a large amount of different bacteria, secondary fermentation is easily happened when the a silo or bale is opened, which causes newly mildewed spots or pieces of mildewed on feeding sections, and when the condition is worse, thorough mildewing and rotting might happen.

### SUMMARY

In view of the above, the present disclosure provides a strain 753 of Lactobacillus rhamnosus and uses thereof, a silage additive and silage. Lactobacillus rhamnosus strain 753 can improve the quality of silage in a high-temperature and high-humidity region, and the silage processed by Lactobacillus rhamnosus strain 753 has good stability and low pH, low aflatoxin B1 content and less dry matter loss. In addition, secondary fermentation can be avoided in the silage processed by Lactobacillus rhamnosus strain 753 when a silo or bale for silage is opened.

In order to solve the technical problems, Lactobacillus rhamnosus strain 753 is provided by the present disclosure, which is preserved in China General Microbiological Culture Collection Center on Jul. 16, 2019, with an accession number of CGMCC 18233 (address: NO.1 West Beichen Road, Institute of Microbiology Chinese Academy of Sciences, Chaoyang District, Beijing 100101, China).

The 16srDNA of Lactobacillus rhamnosus strain 753
is shown as SEQ ID NO. 1:
CTCGCTCCCTAAAAGGGTTACGCCACCGGCTTCGGGTGTTACAAACTCTC
ATGGTGTGACGGGCGGTGTGTACAAGGCCCGGGAACGTATTCACCGCGGC
GTGCTGATCCGCGATTACTAGCGATTCCGACTTCGTGTAGGCGAGTTGCA
GCCTACAGTCCGAACTGAGAATGGCTTTAAGAGATTAGCTTGACCTCGCG
GTCTCGCAACTCGTTGTACCATCCATTGTAGCACGTGTGTAGCCCAGGTC
ATAAGGGGCATGATGATTTGACGTCATCCCCACCTTCCTCCGGTTTGTCA
CCGGCAGTCTTACTAGAGTGCCCAACTAAATGCTGGCAACTAGTCATAAG
GGTTGCGCTCGTTGCGGGACTTAACCCAACATCTCACGACACGAGCTGAC
GACAACCATGCACCACCTGTCATTTTGCCCCCGAAGGGGAAACCTGATCT
CTCAGGTGATCAAAAGATGTCAAGACCTGGTAAGGTTCTTCGCGTTGCTT
CGAATTAAACCACATGCTCCACCGCTTGTGCGGGCCCCCGTCAATTCCTT
TGAGTTTCAACCTTGCGGTCGTACTCCCCAGGCGGAATGCTTAATGCGTT
AGCTGCGGCACTGAAGGGCGGAAACCCTCCAACACCTAGCATTCATCGTT
TACGGCATGGACTACCAGGGTATCTAATCCTGTTCGCTACCCATGCTTTC
GAGCCTCAGCGTCAGTTACAGACCAGACAGCCGCCTTCGCCACTGGTGTT
CTTCCATATATCTACGCATTTCACCGCTACACATGGAGTTCCACTGTCCT
CTTCTGCACTCAAGTTTCCCAGTTTCCGATGCACTTCCTCGGTTAAGCCG
AGGGCTTTCACATCAGACTTAAAAAACCGCCTGCGCTCGCTTTACGCCCA
ATAAATCCGGATAACGCTTGCCACCTACGTATTACCGCGGCTGCTGGCAC
GTAGTTAGCCGTGGCTTTCTGGTTGGATACCGTCACGCCGACAACAGTTA
CTCTGCCGACCATTCTTCTCCAACAACAGAGTTTTACGACCCGAAAGCCT
TCTTCACTCACGCGGCGTTGCTCCATCAGACTTGCGTCCATTGTGGAAGA
TTCCCTACTGCTGCCTCCCGTAGGAGTTTGGGCCGTGTCTCAGTCCCAAT
GTGGCCGATCAACCTCTCAGTTCGGCTACGTATCATTGCCTTGGTGAGCC
GTTACCTCACCAACTAGCTAATACGCCGCGGGTCCATCCAAAAGCGATAG
CTTACGCCATCTTTCAGCCAAGAACCATGCGGTTCTTGGATTTATGCGGT
ATTAGCATCTGTTTCCAAATGTTATCCCCCACTTAAGGGCAGGTTACCCA
CGTGTTACTCACCCGTCCGCCACTCGTTCAAAATTAAATCAAGATGCAAG
CACCTTTCAATAATCAGAACTCGTTCGA.

A silage additive is also provided by the present disclosure, which comprises Lactobacillus rhamnosus strain 753 as an active ingredient.

Preferably, the silage additive is an additive for corn silage.

Preferably, the silage additive is a silage additive for reducing aflatoxin B1 content in silage.

The invention also provides a silage which contains the silage additive.

Preferably, the silage is corn silage.

Preferably, the silage is produced by a method comprising the following steps:

mixing Lactobacillus rhamnosus strain 753 with chopped crop, and

performing fermentation to obtain the silage.

Preferably, the fermentation temperature is 20-45° C.

Preferably, the fermentation conditions are at a temperature of 20-45° C. in the dark with a humidity of 80-85%.

The present disclosure also provides uses of Lactobacillus rhamnosus strain 753 in any one of the followings:

(1) preparing a biological agent for inhibiting the generation of aflatoxin B1;

(2) producing a silage additive; and

(3) producing a silage.

The present disclosure also provides a method of producing a silage comprising:

mixing Lactobacillus rhamnosus with a raw material to obtain a mixture, and

subjecting the mixture to fermentation.

Preferably, the Lactobacillus rhamnosus is Lactobacillus rhamnosus strain 753 which is deposited in China General Microbiological Culture Collection Center with an accession number of CGMCC 18233.

Preferably, the raw material is selected from the group consisting of corn, elephant grass, and rice straw, more preferably, corn.

Preferably, the temperature of the fermentation is 20 to 45° C.

Preferably, the duration of the fermentation is 45 to 90 days.

Compared with the prior art, the present disclosure is described below.

In the present disclosure, according to the potential of low pH growth and high lactic acid yield, Lactobacillus rhamnosus strain 753 is isolated from corn silage, which can effectively improve the quality of silage in high-temperature and high-humidity regions. The effects on whole-plant corn fermentation, aerobic stability of silage and aflatoxin B1 production are studied in the Sichuan area, China. Based on the physiological and biochemical characteristics and 16S rRNA sequencing analysis, the isolated strain is identified as Lactobacillus rhamnosus and named strain 753. After 60 days of fermentation with the treatment of Lactobacillus rhamnosus strain 753, the aflatoxin B1 content can be reduced (as low as 1.19 μg kg⁻¹ Dry Matter (DM)), and lactic acid/acetic acid ratio are also reduced. The silage treated by Lactobacillus rhamnosus strain 753 has good aerobic stability, low pH value (4.88) and low aflatoxin B1 content (1.94 μg kg⁻¹ DM) and less dry matter loss (8.18%) after 5 days aerobic exposure. The counting results of yeast and bacteria (including Escherichia coli) are much lower than control silage after 5 days aerobic exposure under the condition of high temperature and high humidity. In addition, in the silage treated with Lactobacillus rhamnosus strain 753, secondary fermentation can be avoided when the silage is opened. Lactobacillus rhamnosus strain 753 can be used as a candidate strain for silage in tropical and subtropical regions, also for the preparation of biological agents for inhibiting the generation of aflatoxin B1, and for silage additives and silage.

### BRIEF DESCRIPTION OF DRAWINGS

FIG. 1 is an optical microscope image of Lactobacillus rhamnosus strain 753 of the present disclosure.

FIG. 2 is a phylogenetic tree constructed in Example 2.

FIG. 3 is the aerobic stability of the corn silage treated with the strain of Example 4.

FIG. 4 is a graph of aflatoxin (AF) B1 concentration after 60 days of ensiling and 5 days of aerobic exposure in Example 4.

FIG. 5 is a growth curve of lactic acid bacteria under the culture condition of 37° C. in Example 4.

FIG. 6 is a growth curve of lactic acid bacteria under the culture condition of 45° C. in Example 4.

FIG. 7 is a curve showing the acid production of lactic acid bacteria under the culture condition of 37° C. in Example 4.

FIG. 8 is a curve showing the acid production of lactic acid bacteria under the culture condition of 45° C. in Example 4.

### DETAILED DESCRIPTION

In order to make those skilled in the art better understand the technical solution of the present disclosure, the following detailed description of the present disclosure is provided with reference to specific examples.

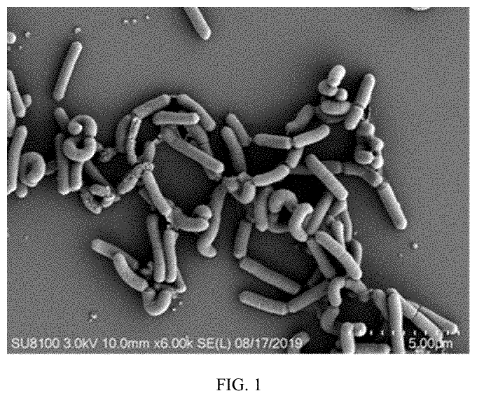

Lactobacillus rhamnosus strain 753 is preserved in China General Microbiological Culture Collection Center on Jul. 16, 2019, with an accession number of CGMCC 18233 (address: NO.1 West Beichen Road, Institute of Microbiology Chinese Academy of Sciences, Chaoyang District, Beijing 100101, China). An optical microscope image of Lactobacillus rhamnosus strain 753 is shown in FIG. 1.

### Example 1

Isolation and Screening of Bacterial Strains

### 1. Raw Materials

Whole corn plants in the milk stage (from three varieties: Yayu 8, Zhongdan 808 and Duoyu 3)

Elephant grass harvested at a height greater than 1.5 m (Cultivar: Guiminyin No. 1)

De Man, Rogosa, Sharpe agar (MRS) broth was prepared by dissolving raw materials (Table 1) in 1 L distilled water, autoclaving at 121° C. for 15 min, and cooling before use.

MRS solid culture medium was prepared by dissolving raw materials (Table 1) in 1 L distilled water, adding 15 g of agar, autoclaving at 121° C. for 15 min, pouring the medium into disposable culture plates, and cooling before use.

TABLE 1
Raw materials of medium

| Materials | Amount | |
|---|---|---|
| Beef extract | 10.00 | g |
| Yeast extract | 5.00 | g |
| Sodium acetate anhydrous | 5.00 | g |
| Peptone | 10.00 | g |
| K₂HPO₄ | 2.00 | g |
| Glucose | 20.00 | g |
| Polysorbate 80 | 1.00 | ml |
| MgSO₄•H₂O | 0.58 | g |
| Ammonium citrate | 2.00 | g |
| MnSO₄•H₂O | 2.00 | g |

### 2. Ensiling Samples

Corn silage sample preparation. Whole plants (from three corn varieties: Yayu 8, Zhongdan 808 and Duoyu 3) in the milk stage were fermented in a large silo (the ensiling conditions of high-temperature and high-humidity area in southwest of China are simulated: temperature 35-45° C., humidity 80-85%), and samples were collected after two months.

Elephant grass silage sample preparation. The Elephant grass (Cultivar: Guiminyin No. 1) was harvested when the height was more than 1.5 m and wrapped into a bale for ensiling (the ensiling conditions of high-temperature and high-humidity area in southwest of China are simulated: temperature 35-45° C., humidity 80-85%), and samples were collected after three months.

### 3. Dilution of Samples

20 g of each silage sample was weighed respectively and put into 180 mL of sterile distilled water, shaken at 4° C. for 1 h, and then continuously diluted in sterile distilled water for 10⁻¹ to 10⁻⁷ dilution respectively. Diluted samples of 10⁻³, 10⁻⁵ and 10⁻⁷ dilutions were plated on solid MRS medium.

### 4. Isolation of Strains

After culturing at 37° C. for 48 h in liquid MRS medium (Land Bridge Science and Technology Co., Ltd., Beijing, China), the lactic acid bacteria were isolated. In order to ensure the accuracy of the lactic acid bacteria community structure in the silage, 20-40 strains were randomly selected from each solid MRS medium sample, and 251 strains were collected in total. Among them, 227 strains were determined to be lactic acid bacteria by gram staining and catalase test, and the 227 strains were stored at −20° C. in a storage tube containing glycerin.

### 5. Primary Screening of Strains

227 strains of lactic acid bacteria obtained by primary screening were inoculated in MRS liquid medium for culture at 37° C., the OD (600 nm) and the pH value were measured at 12 h and 24 h. 30 strains with highest growth efficiency and strongest acid production capability were screened out.

### 6. Re-Screening of Strains

30 strains of lactic acid bacteria obtained by primary screening were inoculated in MRS liquid medium for culture at 45° C., the OD (600 nm) and the pH value were measured at 12 h and 24 h. 3 strains with highest growth efficiency and strongest acid production capability were screened out.

### 7. Screening Results

Best strains at 12 h:

b. Lactobacillus salivarius strain 358(XH 358) having an OD of 0.794 and a pH of 4.02;

c. Lactobacillus rhamnosus strain 753(XH 753) having an OD of 1.241 (highest growth efficiency) and a pH of 3.62 (strongest acid-producing ability);

d. Lactobacillus paracasei strain 761(XH 761) having an OD of 1.034 and a pH of 4.4. Best strains at 24 h:

b. Lactobacillus salivarius strain 358(XH 358) having an OD of 1.164 and a pH of 3.78;

c. Lactobacillus rhamnosus strain 753(XH 753) having an OD of 1.587 (highest growth efficiency) and a pH of 3.69 (strongest acid-producing ability);

d. Lactobacillus paracasei strain 761(XH 761) having an OD of 1.222 and a pH of 3.88.

### Example 2

### Identification of Strains

### Extraction of DNA

Lactobacillus rhamnosus strain 753 was cultured overnight in 5 mL of MRS broth at 37° C. and then the mixture was centrifuged at 10,000×g for 5 min. The cells were washed 2 times with TE buffer (10 mmol L⁻¹ Tri-HCl, 0.1 mmol L⁻¹ EDTA, pH 8.0) in a clean 15 mL microfuge tube and centrifuged again. DNA extraction was performed using a TIANAmp bacterial DNA extraction kit (DP302-02, Tiangen Biotech Co., Ltd., Beijing, China) according to the manufacturer's instructions. The DNA concentration was measured at 260 nm using an ultraviolet-visible spectrophotometer (Shanghai Yangguang Hengping scientific instruments Co., Ltd., Shanghai). DNA extracted from the strain was stored at −20° C. prior to use.

Species Identification by 16S rRNA

The 16S rDNA coding region was amplified through PCR. 1 μL of diluted DNA was used as template for PCR reaction. The PCR primers were 27f (5′-AGAGTTTGATCCTGG CTCAG-3′, SEQ ID NO: 2) and 1492r (5′-TACGGCTACCTTGTTACGACT-3′ SEQ ID NO: 3). The PCR reactions were carried out in 0.2 mL microcentrifuge tubes containing 25 μL of the reaction mixture. All of the PCR reagents were purchased from Tiangen Biochemical Technology Ltd. Primer 27f (0.4 mmol L⁻¹) and primer 1492r (0.4 mmol L⁻¹) were added. The PCR program was: 95° C. for 5 minutes followed by 30 cycles of denaturation at 94° C. for 1 min, annealing at 55° C. for 1 min, extension at 72° C. for 1 min, and then incubation at 72° C. for 5 min. 5 μL of the reaction mixture was analyzed in 1×TBE buffer by 1.5% agarose gel electrophoresis. The gel was stained with ethidium bromide and bands were visualized under UV irradiation. The PCR product was purified using a DNA purification system according to the manufacturer's instructions (Promega, Madison, Wis., USA). 16S rDNA sequence (about 1.5 kb) was subjected to sequence analysis using a 3730xl DNA analyzer (applied biosystems, san Francisco, USA) and the obtained 16S rRNA gene sequence of Lactobacillus rhamnosus 753 was uploaded to GenBank with an accession number of MH333262.

The organism was identified by aligning the 16S rDNA sequence with the 16S rRNA sequences in GenBank using BLAST analysis. Sequence information from representative organisms was introduced into the CLUSTALW program. The 16S rRNA gene sequence of the isolated strain was compared to the sequences of GenBank LAB type strains.

Nucleotide substitution rates were calculated and phylogenetic tree was constructed by the neighbor-joining method (Nei and Saitou, 1987). Bootstrap analysis was performed on 1000 randomly sampled sequences using CLUSTALW software to assess the topology structure of phylogenetic tree. Bacillus subtilis NCDO1769 was used as an exome.

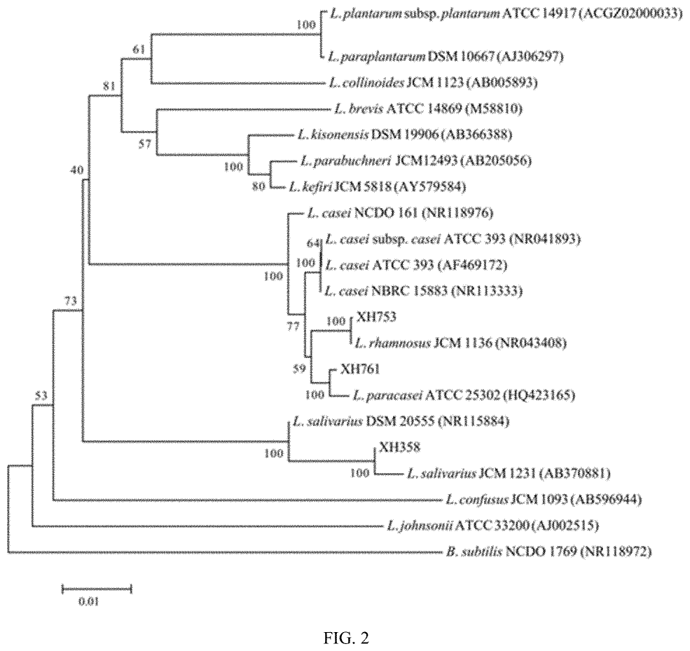

FIG. 2 is a phylogenetic tree showing the relative position of Lactobacillus rhamnosus 753(XH753), which is deduced from the abutment of the complete 16S rDNA sequence. The 1000 copied boot values are displayed on the nodes of the tree. Bacillus subtilis is used as the exome. The phylogenetic tree shows that the relation between Lactobacillus rhamnosus 753(XH753) and Lactobacillus rhamnosus is closest.

The 16S rDNA nucleotide sequence of Lactobacillus rhamnosus 753 is shown in SEQ ID NO: 1, which is deposited in GenBank with an accession number of MH 333262.

### Example 3

### Preparation of Lactobacillus rhamnosus 753, Silage Additive and Silage

Raw materials for ensiling: the whole corn plants in the milk stage were harvested from the test farm of Sichuan Agricultural University (Chongzhou, Sichuan, China)

Test Materials

The test materials included XH358, XH753 and XH761, also four type strains (LP, L. plantarum ATCC 14917; LS, L. salivarius ATCC 11741; LR, L. rhamnosus ATCC 7469 and LPA, L. paracasei ATCC 334), and one commercial LAB inoculant LPC (L. plantarum purchased from Gaofuji, Chengdu, Sichuan, China).

(1) 3 Strains of Lactic Acid Bacteria Through Screening

b. Lactobacillus salivarius 358(XH 358);

c. Lactobacillus rhamnosus 753(XH 753);

d. Lactobacillus paracasei 761(XH 761).

(2) 4 lactobacillus Standard Strains

a′. Lactobacillus plantarum (LP);

b′. Lactobacillus salivarius (LS);

c′. Lactobacillus rhamnosus (LR);

d′. Lactobacillus paracasei (LPA).

The XH753 (Lactobacillus rhamnosus 753), XH358 (Lactobacillus salivarius 358), XH761 (Lactobacillus paracasei 761), LP, LS, LR, LPA and LPC mentioned above were used as silage inoculant.

Each inoculant was dissolved in sterile distilled water as a silage additive in an amount of 10⁸ CFU per gram fresh matter (FM, silage raw materials). The corn (silage raw materials) was chopped into pieces (1-2 cm), and then silage additive was uniformly sprayed on the chopped raw materials to obtain silage. 3 mL of silage additive per 100 g of silage was required. An equal amount of distilled water was used as a control. 1 kg of the corn was packed in a vacuum-sealed polyethylene plastic bag. Each treatment was repeated three times. The bags were stored in a simulated high-temperature and high-humidity dark environment (temperature 35-45° C., humidity 80-85%). After 60 days of ensiling, each treatment sample was opened and the chemical components, the fermentation quality, the microbial community and the aerobic stability of the treated sample were analyzed.

### Example 4

### Detection of Silage

### 1. Chemical and Fermentation Analysis

The corn plants before and after ensiling was dried in a forced air oven at 65° C. for 72 h to determine the Dry Matter (DM) content. The dried samples were ground by a grinder (CT293 Cyclotec™, FOSS Analytical A/S, Hillerød, Denmark) and passed through a 1-mm mesh sieve for future chemical analysis. The crude protein (Avila et al, 2009) content was determined using Kjeldahl method (AOAC, 1990). Neutral detergent fiber (NDF) and acid detergent fiber (ADF) were analyzed using the Ankom 200 system according to the manufacturer's instructions (Ankom technologies, Verporter, N.Y.), and NDF was also analysis using sodium sulfite and alpha-amylase. Water soluble carbohydrates (WSC) was measured by the thracenone-sulphuric acid method (AOAC, 1990). Using 20 g of silage as a material, adding with 180 mL of distilled water into an industrial mixer, and homogenizing for 1 minute to prepare the silage extract. The pH was measured by using a portable pH meter (PHSJ-5; Shanghai LEICI, China). The filtrate was centrifuged at 12,000×g at 4° C. for 10 min, and the supernatant was filtered through a 0.22 μm membrane filter and subjected to organic acid analysis using High Performance Liquid Chromatography (HPLC) equipped with an ultraviolet detector (210 nm) and a column (KC-811, Shimadzu, Kyoto, Japan) at conditions: mobile phase 0.1% H₃PO₄, flow rate 0.5 mL/min, temperature 55° C. For determination of NH₃—N content, 1 mL of trichloroacetic acid (TCA) was added to 4 mL of the filtrate, and the protein was precipitated overnight at 4° C., then centrifuged at 12,000×g for 15 min, and the supernatant was subjected to NH₃—N analysis (Weatherburn, 1967).

### 2. Microbiological Analysis

Silage obtained after 60 days of ensiling was used as a sample, and the microbial composition of the silage was analyzed by a flat plate counting method. 20 g sample was homogenized in a blender with 180 mL of sterile physiological saline (0.85% NaCl) for 1 min. The resultant was subjected to gradient dilution from 10⁻¹ to 10⁻¹⁰. The LAB number was counted on LAB-specific MRS agar (CM 188, Beijing Luqiao, China) at 37° C. for 48 hours under anaerobic conditions. The coliform group was counted on bile salts and crystal violet agar (CM 115, Beijing Luqiao, China) at 37° C. for 24 hours under aerobic condition. The mold and yeast were counted on potato dextrose agar (CM 123, Beijing Luqiao, China) at 25° C. for 4 days.

### 3. Aerobic Stability

Using silage obtained by 60 days on ensiling as a sample, about 800 g of the sample was placed in a separate 2 L insulated container (three repetitions of each treatment), covered with 2 layers of cheesecloth, and stored in a dark environment at a temperature of 35-45° C. and a humidity of 80-85% for 5 days. A data recorder (MT-X, Shenzhen Shenhua science and technology Co., Ltd., China) was inserted into the silage at a depth of 10 cm, and the temperature was measured at 5 minute intervals. DM loss was calculated according to the weight of silage before and after aerobic exposure. The ambient temperature and the temperature in each container were monitored simultaneously for 5 days. The contents of each container were thoroughly mixed and sampled after 5 days of aerobic exposure for analysis of fermentation quality (20 g) and microorganisms (20 g). Aerobic stability was calculated according to Ranjit and Kung (2000) as the number of hours before the temperature of the silage exceeded the baseline ambient temperature by 2° C.

### 4. Aflatoxin B1 Assay

A total of 2 g of the ground sample was weighed into a 50 ml polypropylene tube according to the method of Dogi et al (2013) and Shimsoni et al (2013) and covered with 10 ml of a solvent mixture comprising acetonitrile/water/acetic acid (79:20:1, v/v/v). The sample was left for 5 minutes, centrifuged at 10,000×g for 10 minutes, the supernatant was filtered through a 0.22 μm membrane filter and subjected to AFB1 analysis through a high-performance liquid chromatography column (Cloversil ODS-C18, 150×4.6 mm, 5 μm particle size). Mobile phase (water:acetonitrile:methanol=4:1:1) was pumped at 1.5 ml min⁻¹, the column temperature was 30° C., the injection amount was 20 μl, and the detention time was about 5 min. The detector was a fluorescence detector, the wavelength of excitation wave was 360 nm and the wavelength of emission wave was 440 nm.

### 5. Growth Characteristics of Strains at Different Temperatures

Bacterial liquid cultures were prepared from activated strains, and the bacterial concentrations were adjusted to 10⁸ CFU·ml⁻¹ with U-2910 ultraviolet-visible spectrophotometer. Each strain was set to 3 replicates.

Temperature Resistance Test

The bacterial cultures were inoculated into MRS liquid culture medium (the inoculation proportion taking the 3% volume fraction as a standard). The samples were respectively cultured in an incubator at 4, 10, 20, 30, 40, 45 and 50° C. for 72 h, and then the OD values of the cultures were measured.

### 6. Salt Tolerance Test, Acid and Alkali Resistance Test

Salt Tolerance Test

NaCl was added into MRS liquid medium, wherein the NaCl volume fractions were 3.0% and 6.5% respectively. The bacterial cultures were inoculated into the MRS liquid medium with different NaCl concentrations. The cultures were divided into two groups according to the temperature, 37° C. and 45° C. for 72 h, and then the OD values of the cultures were measured.

Acid and Alkali Resistance Test

Sterile HCl solution and the NaOH solution were used to adjust the pH of the MRS liquid medium and the strains were respectively inoculated into the MRS liquid culture medium with the pH of 3.0, 3.5, 4.0, 4.5, 5.0, 5.5, 6.0, 6.5, 7.0 and 7.5. The cultures were divided into two groups according to the temperature, 37° C. and 45° C. for 72 h, and then the OD values of the cultures were measured.

The OD values in accordance with the growth status are shown in Table 2.

TABLE 2
OD values and corresponding growth status

| OD | Growth Status |
|---|---|
| <0.05 | No growth |
| 0.05-0.1 | Weak growth |
| 0.1-0.5 | Normal Growth |
| >0.5 | Good growth |

### 7. Data Analysis

Statistical analysis was performed using the GLM program of the social science statistical software package (SPSS Version 19.0, SPSS Inc., Chicago, Ill., USA). The chemical composition, fermentation characteristics, microbial count, aflatoxin b 1 and aerobic stability during fermentation were analyzed using one-way analysis of variance (ANOVA). Different sample means were tested using the Turkey Honesty Significance Difference (HSD) test, with p<0.05 being significant.

### 7.1 Characteristics of Lactic Acid Bacteria in Silage

Results are shown in Table 3.

TABLE 3
Characteristics of Lactic Acid Bacteria in Silage

| Strain | XH358 | XH753 | XH761 | LPC |
|---|---|---|---|---|
| Source | Guimu 1 | Yayu 8 | Yayu 8 | Commercial |
| Location of | XH | XH | XH | Unknown |
| Collection | | | | |
| Shape | Rod-shaped | Rod-shaped | Rod-shaped | Rod-shaped |
| Gram staining | + | + | + | + |
| Catalase | − | − | − | − |
| Gas production | + | + | + | − |
| Optical form of | L | L | L | DL |
| lactic acid | | | | |
| Type of | Hetero | Hetero | Hetero | Homo |
| fermentation | | | | |

In Table 3, + indicating that 90% or more of the bacteria were active or positive; − indicating that 90% or more of the bacteria were inactive or negative; w indicating weak activity; homo indicating homofermentation; hetero indicating heterofermentation; LPC, commercial strain of Lactobacillus plantarum.

XH is Xuanhan Huangjin Farm in Dazhou, Sichuan.

7.2 Carbohydrate Fermentation Results by Lactic Acid Bacteria Strains Isolated from Silage

The sugar fermentation characteristics at 37° C. are shown in Table 4.

TABLE 4
Sugar fermentation characteristics at 37° C.

| | Strain | | | | | | | |
|---|---|---|---|---|---|---|---|---|
| Substrate | XH358 | XH753 | XH761 | LP | LS | LR | LPA | LPC |
| Galactose | + | + | + | + | + | + | + | + |
| Inulin | + | W | − | + | W | + | + | + |
| Raffinose | + | + | W | + | + | + | + | + |
| D-Trehalose | + | + | + | + | + | + | + | + |
| D-Glucose | + | + | + | + | + | + | + | + |
| Maltose | + | + | + | + | + | + | + | + |
| Sorbitol | − | + | + | + | + | + | + | + |
| D-Mannitol | − | + | + | + | + | + | + | + |
| Melibiose | + | + | + | + | + | − | + | + |
| Rhamnose | − | W | + | − | + | − | − | − |
| Lactose | + | + | W | + | + | + | + | + |
| Salicin | W | + | + | + | + | + | + | + |
| Arabinose | + | + | + | + | − | + | + | + |
| Saccharose | + | + | + | + | + | + | + | + |
| D-Mannose | + | + | + | + | + | + | + | + |
| Esculin | − | + | + | + | + | + | + | + |
| D-Fructose | + | + | + | + | + | + | + | + |
| Cellobiose | − | + | + | + | − | + | + | + |
| Semi-solid agar | − | + | + | + | + | + | + | + |
| Xylose | + | + | − | − | + | − | − | − |
| Hippuric acid | + | + | − | − | − | + | − | − |
| Amygdalin | W | W | − | + | W | + | + | W |

The sugar fermentation characteristics at 45° C. are shown in Table 5.

TABLE 5
Sugar fermentation characteristics at 45° C.

| | Strain | | | | | | | |
|---|---|---|---|---|---|---|---|---|
| Substrate | XH358 | XH753 | XH761 | LP | LS | LR | LPA | LPC |
| Galactose | + | + | + | + | + | + | + | + |
| Inulin | + | W | + | + | + | + | + | + |
| Raffinose | + | + | W | + | + | + | + | + |
| Mushroom | + | + | + | + | + | + | + | + |
| candy | | | | | | | | |
| Glucose | + | + | + | + | + | + | + | + |
| Maltose | + | + | + | + | + | + | + | + |
| Sorbitol | − | + | + | + | + | + | + | + |
| Sorbitol | − | + | + | + | + | + | + | + |
| Melibiose | + | + | + | + | + | − | + | + |
| L-rhamnose | − | W | + | − | + | − | − | − |
| Salicin | W | + | + | + | + | + | + | + |
| Arabinose | + | + | + | + | − | + | + | + |
| Sucrose | + | + | + | + | + | + | + | + |
| Mannose | + | + | + | + | + | + | + | + |
| Aesculin | − | + | + | + | + | + | + | + |
| Fructose | + | + | + | + | + | + | + | + |
| 1% NaCl | | + | + | + | − | + | + | + |
| Salicylin | | | | | | | | |
| D(+)- | − | + | + | + | + | + | + | + |
| Cellobiose | | | | | | | | |
| Agar | + | + | − | − | + | − | − | − |
| Xylose | + | − | − | + | − | − | + | − |
| Lactose | + | + | + | + | + | + | + | + |
| Hippuric | W | + | + | + | + | + | W | + |
| acid | | | | | | | | |
| Amygdalin | + | + | − | + | + | + | − | + |

In the above tables, + indicating that 90% and more bacteria can ferment the substance; − indicating that 90% and above of the strains cannot ferment the substance; w indicating small amounts of the substance can be fermented; LP, Lactobacillus plantarum; LS, Lactobacillus salivarius; LR, Lactobacillus rhamnosus; LPA, Lactobacillus paracasei; LPC, commercial strain of Lactobacillus plantarum.

Tables 4 and 5 show that isolated Lactobacillus rhamnosus strain 753(M-1753) and LPC can grow in medium containing 6.5% NaCl. All strains can ferment with galactose, D-trehalose, D-glucose, maltose, mannose, arabinose, sucrose and D-fructose, but cannot effectively use hippuric acid.

7.3 Chemical Composition, Microbial Composition and Aflatoxin B1 (AFB1) Content in Corn Plant before Ensiling

The silage raw material, fresh cut whole corn plants, contained 28.38% DM and the concentrations of CP, NDF, ADF, and WSC were 5.93%, 52.15%, 30.53% and 17.77%, respectively. The concentration of AFB1 was 1.03 μg/kg DM. The amounts of LAB, Escherichia coli, yeast and mold were 4.37, 7.34, 5.13 and 1.27 log₁₀ of CFU/g. Results are shown in Table 6.

TABLE 6

| | |
|---|---|
| DM Content (%) | 28.38 |
| pH | 5.78 |
| Crude protein (% DM) | 5.93 |
| Neutral detergent fiber (% DM) | 52.15 |
| Acid detergent fiber (% DM) | 30.53 |
| Water-soluble carbohydrates (% DM) | 17.77 |
| Aflatoxin B1 (μg kg⁻¹DM) | 1.03 |
| Lactobacillus (log₁₀ cfu g⁻¹ FM) | 4.37 |
| Escherichia coli (log₁₀ cfu g⁻¹ FM) | 7.34 |
| Yeast (log₁₀ cfu g⁻¹ FM) | 5.13 |
| Mold (log₁₀ cfu g⁻¹ FM) | 1.27 |

DM, dry matter; FM, fresh matter; CFU, colony forming unit.

7.4 Chemical Composition, Fermentation Characteristics and Microbial Count Results of Corn Silage Obtained after 60 Days of Silage Under High Temperature and High Humidity Conditions

The high-temperature and high-humidity conditions are as follows: the temperature is 35-45° C., and the humidity is 80-85%, so as to simulate the high-temperature and high-humidity region in southwest China. The results are shown in Table 7.

TABLE 7

| Strain | Control | XH358 | XH753 | XH761 | LP | LS | LR | LPA | LPC | SEM |
|---|---|---|---|---|---|---|---|---|---|---|
| pH | 4.03a | 3.85b | 3.85b | 3.87b | 3.91ab | 3.81b | 3.87b | 3.79b | 3.89ab | 0.116 |
| DM (%) | 26.41b | 26.27ab | 26.51ab | 27.06a | 26.09bc | 26.52ab | 25.20d | 25.82c | 25.65cd | 0.261 |
| DM loss | 2.96d | 3.75ab | 3.46bc | 2.89de | 2.97d | 3.39c | 3.80a | 3.31c | 3.39c | 0.149 |

Chemical Composition, % of DM

| | | | | | | | | | | |
|---|---|---|---|---|---|---|---|---|---|---|
| Crude protein | 6.91cd | 7.35ab | 6.43e | 6.95c | 7.00c | 6.66de | 7.45a | 7.56a | 7.50a | 0.093 |
| NDF | 54.61d | 55.07cd | 58.92a | 53.17d | 55.68bcd | 52.88d | 54.49ab | 57.96abe | 55.49bcd | 1.404 |
| ADF | 30.12bcd | 30.83abcd | 33.109a | 27.01e | 29.32cde | 28.46de | 32.07ab | 32.27ab | 29.93bcd | 1.072 |
| WSC | 1.67ef | 1.86def | 1.48f | 2.45bc | 2.441bc | 2.065cde | 3.10a | 2.373bcd | 2.774b | 5.393 |

Fermentation product, mg g⁻¹ DM

| | | | | | | | | | | |
|---|---|---|---|---|---|---|---|---|---|---|
| Lactic acid | 70.30bc | 70.53bc | 58.93d | 63.15cd | 72.66b | 70.82bc | 75.39b | 77.68b | 47.10e | 2.815 |
| Acetic acid | 11.97c | 19.01b | 34.49a | 20.67b | 14.98c | 15.58c | 12.43c | 13.41c | 8.33d | 1.548 |
| LA/AA | 5.87ab | 3.71cd | 1.70d | 3.05bcd | 6.25a | 4.54bcd | 6.13a | 5.80ab | 5.63ab | 0.718 |
| NH₃—N (%) | 6.90a | 4.05d | 2.39e | 6.14b | 5.49bc | 5.29c | 3.90d | 2.58e | 4.18d | 1.358 |

Microorganism, log₁₀ CFU g⁻¹ FM

| | | | | | | | | | | |
|---|---|---|---|---|---|---|---|---|---|---|
| LAB | 6.34bc | 6.66a | 6.06d | 6.03d | 6.09d | 6.39bc | 6.15cd | 6.48ab | 6.35bc | 0.106 |
| Mold | <1.00 | ND | ND | <1.00 | ND | ND | ND | <1.00 | ND | NA |
| Yeast | 1.34c | 1.93b | ND | ND | 1.32c | 2.02b | 2.18a | 0.59e | l.lOd | 0.59 |

| | | |
|---|---|---|
| E. coli | ND | NA |

In the table, a-f data are means of three samples, means in the same column followed by different letters differ (P<0.05).

FM, fresh matter; DM, dry matter; NDF, neutral detergent fiber; ADF, acid detergent fiber; WSC, water soluble carbohydrate; LAB, lactic acid bacteria; ND is not detected, NA is not applicable; LP, Lactobacillus plantarum; LS, Lactobacillus salivarius; LR, Lactobacillus rhamnosus; LPA, Lactobacillus paracasei; LPC, commercial Lactobacillus plantarum; SEM, standard error of the mean. Control is the sample treated with the same volume of distilled water.

WSC content in the sample treated by Lactobacillus rhamnosus 753 (XH753) is the lowest (P<0.05). Compared with other groups, the concentration of acetic acid (AA) in the XH753 treated corn silage is relatively high and reaches 34.49 mg/DM(g). Therefore, the LA/AA ratio is also the lowest in the Lactobacillus rhamnosus 753(XH753) group. Control group has the highest NH₃—N content (total nitrogen content), while the Lactobacillus rhamnosus 753 (XH753) group and LPA group are the lowest (P<0.05).

7.5 PH, DM Loss and Microbial Count in Corn Silage after 5 Days of Aerobic Exposure under High Temperature, High Humidity Conditions

Aerobic exposure was carried out after 60 days of ensiling under high temperature and high humidity conditions for 5 days. The results are shown in Table 8.

The high-temperature and high-humidity conditions are as follows: the temperature is 35-45° C., and the humidity is 80-85%, so as to simulate the high-temperature and high-humidity region in southwest China.

TABLE 8

| Strain | Control | XH358 | XH753 | XH761 | LP | LS | LR | LPA | LPC | SEM |
|---|---|---|---|---|---|---|---|---|---|---|
| pH | 6.02b | 5.38d | 4.88e | 5.65c | 5.98b | 5.93b | 6.39a | 5.63c | 6.54a | 0.884 |
| DM loss | 10.23b | 9.51c | 8.18d | 10.65b | 12.62a | 10.54b | 9.25c | 9.30c | 12.92a | 0.253 |

Microorganism, log₁₀ CFU g⁻¹ FM

| | | | | | | | | | | |
|---|---|---|---|---|---|---|---|---|---|---|
| LAB | 7.57c | 7.08e | 7.94a | 7.97a | 7.85b | 7.03e | 7.47d | 7.61c | 7.90ab | 0.422 |
| Mold | ND | ND | ND | ND | ND | ND | ND | ND | ND | NA |
| Yeast | 6.79a | 4.18d | 3.63e | 4.24d | 5.96b | 6.72a | 6.87a | 6.80a | 4.89c | 0.111 |
| E. coli | 4.44e | 2.49f | 2.02g | 2.49f | 4.44e | 5.50d | 6.39b | 4.44e | 7.30a | 2.052 |

In the table, a-f data are means of three samples, means in the same column followed by different letters differ (P<0.05).

DM, dry matter; LP, Lactobacillus plantarum; LS, Lactobacillus salivarius; LR, Lactobacillus rhamnosus; LPA, Lactobacillus paracasei; LPC, commercial Lactobacillus plantarum; SEM, standard error of the mean.

All mold, yeast and E. coli colonies are drastically reduced after ensiling. LAB strains are dominant species in fermentation, ranging from 6 to 7 log₁₀ CFU g⁻¹FM. Yeasts remain below 2 log₁₀ CFU g⁻¹FM. Neither mold nor E. coli is detected in all silage.

### 7.6 Aerobic Stability of Corn Silage Under High Temperature and High Humidity Conditions

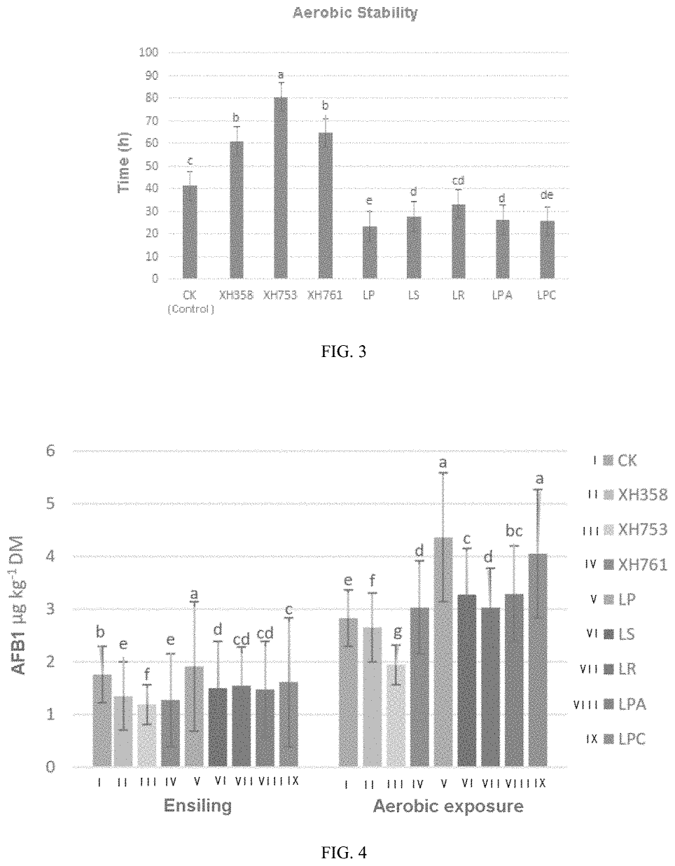

The results are shown in FIG. 3.

In FIG. 3, a-e data are means of three samples, means in the same column followed by different letters differ (P<0.05). LP, Lactobacillus plantarum; LS, Lactobacillus salivarius; LR, Lactobacillus rhamnosus; LPA, Lactobacillus paracasei; LPC, commercial Lactobacillus plantarum; SEM, standard error of the mean.

As shown in tables 7, 8 and FIG. 3, silage treated with Lactobacillus rhamnosus 753 (XH753) of the present disclosure shows relatively strong aerobic stability, the highest LAB amount and lowest yeast and coliform population, as well as the lowest pH and DM loss (P<0.05).

7.7 Aflatoxin (AF) B1 Concentration after 60 Days of Ensiling and 5 Days of Aerobic Exposure

The results are shown in FIG. 4.

In FIG. 4, a-g data are means of three samples, means in the same column followed by different letters differ (P<0.05). LP, Lactobacillus plantarum; LS, Lactobacillus salivarius; LR, Lactobacillus rhamnosus; LPA, Lactobacillus paracasei; LPC, commercial Lactobacillus plantarum; SEM, standard error of the mean.

FIG. 4 shows the concentration of AFB1 after 60 days of ensiling and 5 days of air exposure. Compared with fresh material (having AFB1 at a concentration of 1.03 μg kg⁻¹DM), the AFB1 in all samples increased both during ensiling and during air exposure, with higher values observed after 5 days of air exposure. After 60 days of ensiling, the AFB1 concentration in Lactobacillus rhamnosus 753 (XH753) group was the lowest, while the AFB1 concentration in LP-treated silage was the highest (P<0.05). After 5 days of air exposure, except for the Lactobacillus rhamnosus 753 (X11753) treated group (<2 μg kg⁻¹DM), the AFB1 concentrations in all other silage increased dramatically. In addition, the LP and LPC treatment groups had the highest AFB1, exceeding 4 μg kg⁻¹DM.

### 7.8 Temperature Resistance Characteristics

TABLE 9
Temperature resistance characteristics

| Item | Treatment | LP | LS | LR | LPA | XH358 | XH753 | XH761 | LPC |
|---|---|---|---|---|---|---|---|---|---|
| Different | 4° C. | W | W | − | W | W | − | − | W |
| temperature | 10° C. | W | W | W | W | W | W | − | W |
| test | 20° C. | + | + | + | + | + | + | + | + |
| | 30° C. | + | + | + | + | + | + | + | + |
| | 40° C. | + | + | + | + | + | + | + | + |
| | 45° C. | − | + | W | − | + | + | + | W |
| | 50° C. | − | − | W | − | W | W | W | W |

Lactobacillus rhamnosus 753 of the present disclosure grows well under the culture condition of 20-45° C., showing better high temperature resistance.

### 7.9 Salt Tolerance, Acid and Alkali Resistance

The salt tolerance, acid and alkali resistance characteristics under the culture conditions at 37° C. are shown in Table 10.

TABLE 10
Salt tolerance, acid and alkali resistance characteristics (37° C.)

| Item | Treatment | LP | LS | LR | LPA | XH358 | XH753 | XH761 | LPC |
|---|---|---|---|---|---|---|---|---|---|
| Salt | 3% NaCl | + | + | + | + | + | + | + | + |
| tolerance | 6.5% NaCl | W | − | + | + | − | + | − | + |
| test | | | | | | | | | |
| Acid | pH = 3 | − | − | − | − | − | − | − | − |
| resistance | pH = 3.5 | W | − | + | W | + | W | + | W |
| test | pH = 4 | W | W | + | W | + | + | + | + |
| | pH = 4.5 | + | + | + | + | + | + | + | + |
| | pH = 5 | + | + | + | + | + | + | + | + |
| | pH = 5.5 | + | + | + | + | + | + | + | + |
| | pH = 6 | + | + | + | + | + | + | + | + |
| | pH = 7 | + | + | + | + | + | + | + | + |
| | pH = 7.5 | + | + | + | + | + | + | + | + |

Under the culture condition of 37° C., Lactobacillus rhamnosus 753 of the present disclosure can grow well in a liquid culture medium with NaCl concentration of 3.0-6.5%, or a liquid culture medium with a pH of 4-7.5.

The salt tolerance, acid and alkali resistance characteristics under the culture conditions at 45° C. are shown in Table 11.

TABLE 11
Salt tolerance, acid and alkali resistance characteristics (45° C.)

| Item | Treatment | LP | LS | LR | LPA | XH358 | XH753 | XH761 | LPC |
|---|---|---|---|---|---|---|---|---|---|
| Salt | 3% NaCl | + | + | + | + | + | + | + | + |
| tolerance | 6.5% NaCl | W | − | + | + | − | + | − | + |
| test | | | | | | | | | |
| Acid | pH = 3 | − | − | − | − | − | − | − | − |
| resistance | pH = 3.5 | W | − | + | W | + | W | + | W |
| test | pH = 4 | W | W | + | W | + | + | + | + |
| | pH = 4.5 | + | + | + | + | + | + | + | + |
| | pH = 5 | + | + | + | + | + | + | + | + |
| | pH = 5.5 | + | + | + | + | + | + | + | + |
| | pH = 6 | + | + | + | + | + | + | + | + |
| | pH = 7 | + | + | + | + | + | + | + | + |
| | pH = 7.5 | + | + | + | + | + | + | + | + |

Under the culture condition of 45° C., Lactobacillus rhamnosus 753 of the present disclosure can grow well in a liquid culture medium with NaCl concentration of 3.0-6.5%, or a liquid culture medium with a pH of 4-7.5.

### 7.10 Growth Rate of Strains

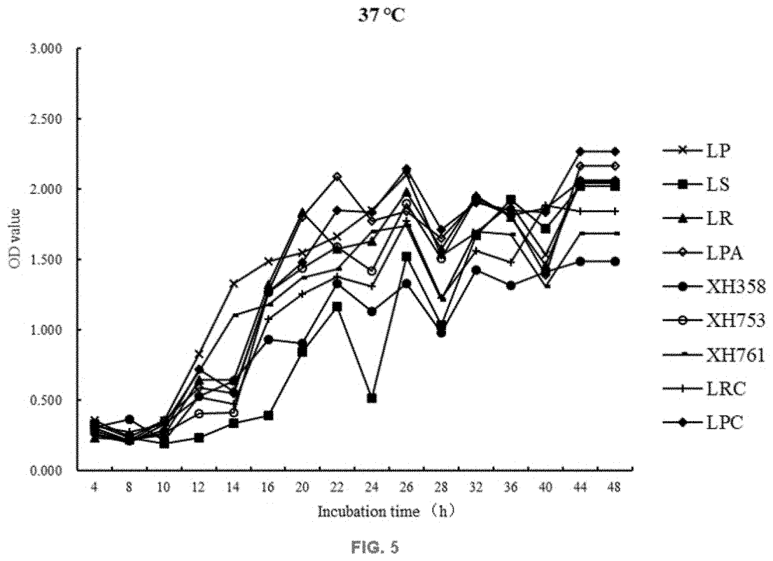

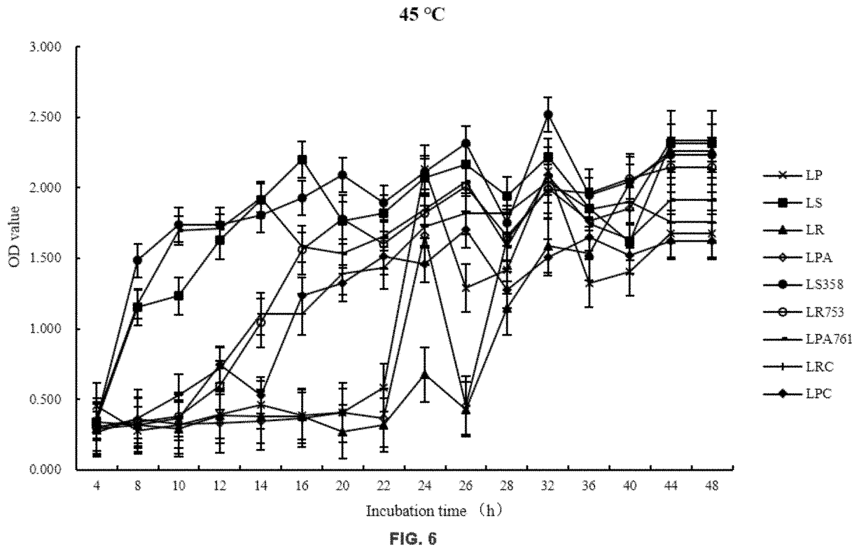

The OD values of the lactic acid bacteria strains at 37° C. and 45° C. were measured, and the results are shown in FIG. 5 and FIG. 6.

FIG. 5 and FIG. 6 are growth curves of lactic acid bacteria under the culture condition of 37° C. and 45° C., respectively.

As shown in FIG. 5 and FIG. 6, the growth curves of the three selected strains: Lactobacillus salivarius 358, Lactobacillus rhamnosus 753 and Lactobacillus paracasei 761, are different at 37° C. and 45° C. compared with the standard strains. At 37° C., the growth rates of these three strains reach the maximum at 10 h of culture, and grow into a stationary phase at about 20 h. OD values of strains XH358, XH753 and XH761 are 0.904, 1.438 and 1.373, respectively, after 20 h fermentation. The growth rate of XH753 is the fastest and more stable within 24 h, the growth rate of XH761 is in the middle, and the growth rate of XH358 is the slowest. At 45° C., all three strains grow better than the standard strains. The growth rate of XH358 reaches maximum at 8 h, and the OD value stably increases from 10 h. The growth rate of XH753 reaches maximum at 12 h, and enters stationary phase starting at 16 h. XH761 reaches a maximum growth rate at 8 h, and enters stationary phase starting at 10 h.

### 7.11 Acid Production of Strains

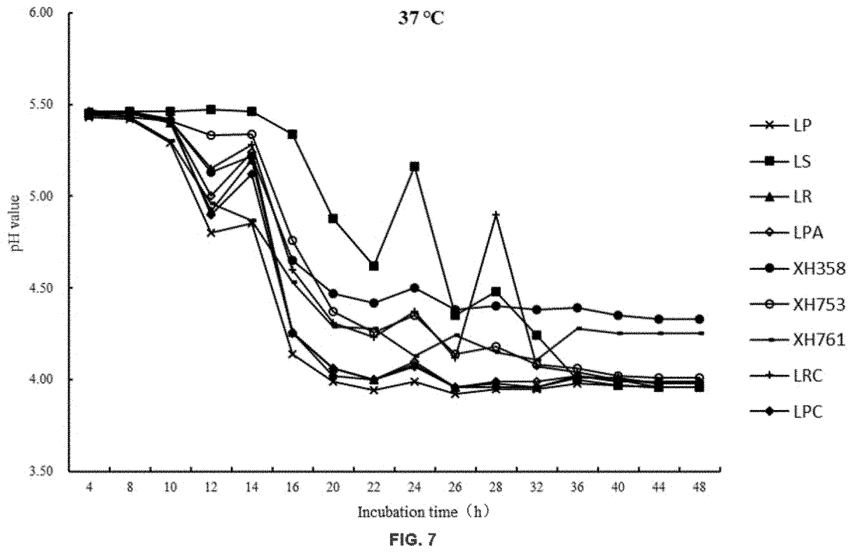

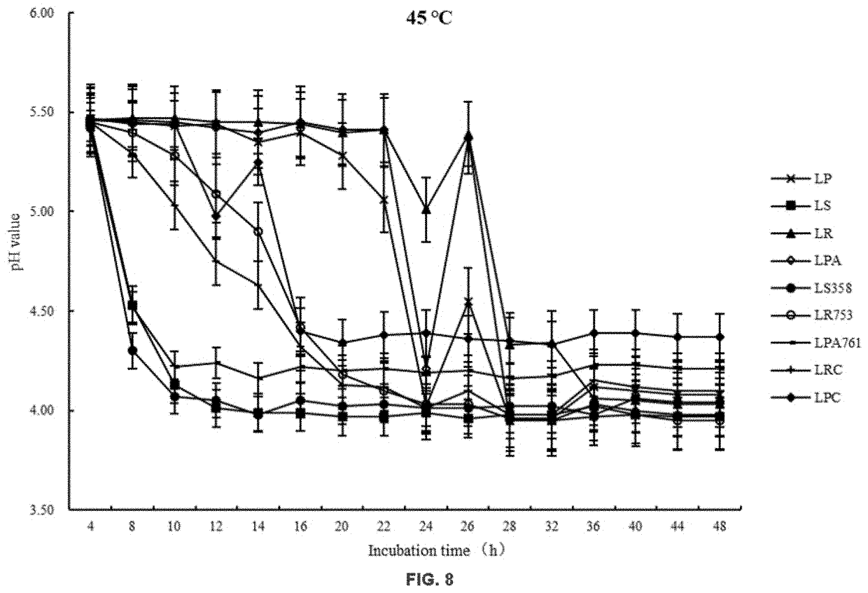

The pH of the liquid medium was measured under the culture conditions of 37° C. and 45° C. and the results are shown in FIG. 7 and FIG. 8.

FIG. 7 and FIG. 8 are curves showing the acid production of lactic acid bacteria under the culture condition of 37° C. and 45° C., respectively.

As shown in FIG. 7 and FIG. 8, the pH values of all the liquid medium at 37° C. and 45° C. tend to decrease overall from 5.5 and keep stable at around 4.3. XH358 and XH753 have a maximum acid production rate at 14 h and enter stationary phase at about 20 h. In XH761 culture, the pH decreases the most at 10 h and enters stationary phase at 20 h. At 45° C., the acid production rates of XH358 and XH761 reach the maximum in about 8 hours, and enter stationary phase at about 10 h. In XH753 culture, the pH decreases the fastest at 14 h, and enters stationary phase at around 24 h.

Result Analysis

A phylogenetic tree according to the 16S rDNA sequence of some LAB strains is shown in FIG. 1. Strain XH358 strain has a sequence completely matched with that of Lactobacillus salivarius. Strain XH753 is closely related to Lactobacillus rhamnosus, and their 16S rDNA gene sequences have 100% similarity. In addition, XH761 is most closely related to Lactobacillus paracasei, with 100% similarity in the 16S rDNA gene sequence.

Through the analysis of chemical components, no significant relation between the strain and the quality of the corn silage is found. However, under high temperature and high humidity conditions, the aerobic stability of corn silage treated by Lactobacillus plantarum poor, whereas corn silage treated by Lactobacillus rhamnosus 753 of the present disclosure has an improved aerobic stability, low pH, less DM loss, and less yeast and Escherichia coli. Under natural conditions, the corns are usually contaminated with AFB1 and no concentration change occurs after ensiling. In the silage treated with Lactobacillus rhamnosus 753 and after aerobic exposure, the increase of AFB1 is the less, which indicates that Lactobacillus rhamnosus 753 has a potential role in inhibiting the growth of fungi and the generation of AFB1.

In conclusion, in tropical or subtropical regions, Lactobacillus plantarum is not recommended to be used as a whole-plant corn silage additive, while Lactobacillus rhamnosus 753 may be a candidate strain to be used to improve aerobic stability of corn silage and inhibit the accumulation of AFB1 in corn silage in high-temperature and humid regions.

The above is only a preferred embodiment of the present disclosure, and it should be noted that the above preferred embodiment should not be considered as limiting the present invention, and the protection scope of the present invention should be subject to the scope defined by the claims. It will be apparent to those skilled in the art that various modifications and adaptations can be made without departing from the spirit and scope of the invention, and these modifications and adaptations should be considered within the scope of the invention.

What is claimed is:

1. A method of producing a silage comprising mixing Lactobacillus rhamnosus with a raw material to obtain a mixture, and subjecting the mixture to fermentation.

2. The method according to claim 1, wherein the Lactobacillus rhamnosus is Lactobacillus rhamnosus strain 753 which is deposited in China General Microbiological Culture Collection Center with an accession number of CGMCC 18233.

3. The method according to claim 1, wherein the raw material is selected from the group consisting of corn, elephant grass, and rice straw.

4. The method according to claim 3, wherein the raw material is corn.

5. The method according to claim 1, wherein the temperature of the fermentation is 20 to 45° C.

6. The method according to claim 1, wherein the duration of the fermentation is 45 to 90 days.

7. The method according to claim 1, wherein the raw material is chopped before mixing with Lactobacillus rhamnosus.
